(19) United States
(12) Patent Application Publication
(10) Pub. No.: US 2024/0071614 A1
(43) Pub. Date: Feb. 29, 2024

(54) SERVER, METHOD AND PROGRAM FOR PROVIDING HEALTH CARE SERVICES

(71) Applicant: INDUSTRY-ACADEMIC COOPERATION FOUNDATION, YONSEI UNIVERSITY, Seoul (KR)

(72) Inventors: Dong Wook KIM, Seoul (KR); Jinsung KIM, Seoul (KR); Hwa Kyung BYUN, Seoul (KR)

(73) Assignee: INDUSTRY-ACADEMIC COOPERATION FOUNDATION, YONSEI UNIVERSITY, Seoul (KR)

(21) Appl. No.: 18/454,574

(22) Filed: Aug. 23, 2023

(30) Foreign Application Priority Data
Aug. 24, 2022 (KR) 10-2022-0105874
Aug. 24, 2022 (KR) 10-2022-0105875

(51) Int. Cl. G16H 40/67; A61B 5/00; A61B 5/0205; A61B 5/318; G16H 70/20

(52) U.S. Cl. CPC G16H 40/67; A61B 5/02055; A61B 5/318; A61B 5/4812; A61B 5/6833; G16H 70/20; A61B 5/024

(57) ABSTRACT

The inventive concept may transmit an analysis result to at least one of the user's terminal, the medical staffs terminal, and the external server which has generated a request when a command for transmitting the analysis result is requested from at least one of the user's terminal, the medical staffs terminal, and the external server.

### CROSS-REFERENCE TO RELATED APPLICATIONS

A claim for priority under 35 U.S.C. § 119 is made to Korean Patent Application Nos. 10-2022-0105875 filed on Aug. 24, 2022 and 10-2022-0105874 filed on Aug. 24, 2022 in the Korean Intellectual Property Office, the entire contents of which are hereby incorporated by reference.

### BACKGROUND

Embodiments of the inventive concept described herein relate to a server, method, and program for providing health care services.

Conventionally, a portable sleep analysis device using an accelerometer and an optical plethysmograph is convenient, but has limitations due to poor accuracy.

In other words, the conventional portable sleep analysis device records total sleep time, deep sleep time, sleep efficiency, or the like by using a sensor (smart watch) worn on a user's wrist during sleep and shows the results of analysis of the time the user went to bed, the time the user actually slept, the REM sleep and non-REM sleep cycles, and the like.

However, the conventional portable sleep analysis device using an accelerometer and an optical plethysmograph measures a patient's movement through the accelerometer to determine whether the patient is asleep, and identifies a heart rate and a heart rate variability through the optical plethysmograph to determine the depth of sleep. However, the accuracy was relatively low compared to sleep analysis through direct measurement of electrocardiogram and EEG.

On the other hand, sleep analysis through electrocardiogram measurement and EEG measurement, which are carried out in neuroscience research institutes, is capable of precise measurement and analysis, but is inconvenient because measurement cannot be easily performed at home such as a smart watch.

In addition, when a tumor occurs in the lung or liver, radiation therapy is performed to remove the tumor. While a patient receiving radiation therapy is breathing, a region to be treated with radiation moves, which lowers the accuracy of radiation therapy and causes unnecessary exposure. That is, the tumor cannot be accurately irradiated, and other normal parts are exposed to radiation, resulting in further damage.

To prevent radiation exposure due to unnecessary exposure and improve the effect of radiation therapy, the patient has a regular breathing of a certain cycle, and the respiratory-gated radiation therapy (RGRT) is performed according to the cycle. To perform the respiratory-gated radiation therapy, first, the patient needs to be trained to breathe regularly in a certain cycle.

To this end, a number of breathing exercise devices have been proposed that make the patient breathe regularly in a certain cycle.

However, the conventional respiratory training device has a large scale of system for monitoring, requires additional manpower to operate the system and a specific facility space where the system itself is installed, so it is not applicable in most medical facilities despite the effect of breathing exercise.

Therefore, in recent years, research on improved health management services that are easy to carry such that patients or subjects interested in breathing exercise can do breathing exercise on their own when they want, and that can immediately notify when a change in breathing cycle occurs has been continuously conducted.

### SUMMARY

Objects of embodiments disclosed herein are to provide an analysis service for a sleep state for each user.

Furthermore, objects of embodiments disclosed herein are to provide a customized service for improving a sleep state for each user.

Furthermore, objects of embodiments disclosed herein are to improve the precision of sleep monitoring.

Furthermore, objects of embodiments disclosed herein are to monitor breathing during sleep.

Furthermore, objects of embodiments disclosed herein are to reduce accidents due to sleep apnea.

Furthermore, objects of embodiments disclosed herein are to measure and record various bio-signals in real time.

Furthermore, objects of embodiments disclosed herein are to improve the quality of sleep through real-time biosignal analysis.

Furthermore, objects of embodiments disclosed herein are to improve the quality of sleep through biosignal analysis using AI.

Furthermore, objects of embodiments disclosed herein are to provide an analysis service for breathing exercise for each user.

Furthermore, objects of embodiments disclosed herein are to provide an analysis service of heart rate according to breathing exercise for each user.

Furthermore, objects of embodiments disclosed herein are to provide an analysis service of heart rate for each body temperature according to breathing exercise for each user.

Furthermore, objects of embodiments disclosed herein are to provide a customized service for providing a guide for improving breathing exercise for each user.

Furthermore, objects of embodiments disclosed herein are to provide a customized service for providing a guide for improving a heart rate according to breathing exercise for each user.

Furthermore, objects of embodiments disclosed herein are to provide a customized service for providing a guide for improving a heart rate for each body temperature according to breathing exercise for each user.

However, problems to be solved by the inventive concept may not be limited to the above-described problems. Although not described herein, other problems to be solved by the inventive concept can be clearly understood by those skilled in the art from the following description.

According to an embodiment, a server for providing health care services includes a communication unit that communicates with at least one of a user's terminal, a medical staffs terminal related to the user's terminal, an external server related to the user's terminal, and a patch-type sensor, and a processor that controls an operation related to provision of the health care services, wherein the processor may receive a biological signal of a sleeping user measured by the patch-type sensor, determine whether a sleep state of the user satisfies a preset sleep state condition for a preset time period as an analysis result of analyzing the biological signal through an artificial intelligence model, store the analysis result for the preset time period when the sleep state of the user does not satisfy the sleep state condition, generate a request to transmit the analysis result to at least one of the user's terminal, the medical staffs terminal, and the external server when the storing of the analysis result is completed, and transmit the analysis result to at least one of the user's terminal, the medical staffs terminal, and the external server which has generated the request when a command for transmitting the analysis result is requested from at least one of the user's terminal, the medical staffs terminal, and the external server.

Furthermore, the processor may further generate a request to transmit guide information for improving the sleep state of the user according to the analysis result to at least one of the user's terminal, the medical staffs terminal, and the external server which has generated a request when the command for transmitting the analysis result is requested, and further transmit the guide information to at least one of the user's terminal, the medical staffs terminal, and the external server which has generated a request when a command for transmitting the guide information is requested from at least one of the user's terminal, the medical staffs terminal, and the external server which has generated a request.

Furthermore, the processor may determine whether a degree of breathing of the user satisfies a preset breathing level for the preset time period.

Furthermore, the processor may determine whether a degree of a heart rate of the user satisfies a preset heart rate level for the preset time period.

Furthermore, the processor may determine whether a degree of electrocardiogram of the user satisfies a preset electrocardiogram level for the preset time period.

Furthermore, the processor may determine whether a degree of a body temperature of the user satisfies a preset body temperature level for the preset time period.

Furthermore, the processor may determine whether a degree of a sleeping posture of the user satisfies a preset sleeping posture level for the preset time period.

Furthermore, the communication unit may further perform communication with a virtual reality device, and the processor may receive breathing exercise information of the user who uses the virtual reality device measured by a magnetic sensor of the patch-type sensor, determine whether a degree of breathing exercise of the user satisfies a preset breathing exercise condition for the preset time period as a result of analyzing the breathing exercise information through the artificial intelligence model, store a breathing exercise analysis result for the preset time period when the degree of the breathing exercise of the user does not satisfy the breathing exercise condition, generate a request to transmit the breathing exercise analysis result to at least one of the user's terminal, the medical staff's terminal related to the user's terminal, and the external server related to the user's terminal when storing of the breathing exercise analysis result is completed, and transmit the breathing exercise analysis result to at least one of the user's terminal, the medical staffs terminal, and the external server which had generated the request when a command for transmitting the breathing exercise analysis result is requested from at least one of the user's terminal, the medical staffs terminal, and the external server.

Furthermore, the processor may further generate a request to transmit first exercise guide information for strengthening a lung condition of the user according to the breathing exercise analysis result to at least one of the user's terminal, the medical staffs terminal, and the external server which had generated the request when the command for transmitting the breathing exercise analysis result is requested, and further transmit the first exercise guide information to at least one of the user's terminal, the medical staffs terminal, and the external server which had generated a request when a command for transmitting the first exercise guide information is requested from at least one of the user's terminal, the medical staffs terminal, and the external server which had generated the request.

Furthermore, the processor may further receive heart rate information of the user using the virtual reality device measured by a heart rate sensor of the patch-type sensor, further determine whether a degree of heart rate of the user satisfies a preset heart rate level for the preset time period as a result of further analyzing the heart rate information through the artificial intelligence model, further store a heart rate analysis result for the preset time period when the degree of heart rate of the user does not satisfy the heart rate level, further generate a request to transmit the heart rate analysis result to at least one of the user's terminal, the medical staff's terminal related to the user's terminal, and the external server related to the user's terminal when storing of the heart rate analysis result is completed, and transmit the heart rate analysis result to at least one of the user's terminal, the medical staffs terminal, and the external server which had generated the request when a command for transmitting the heart rate analysis result is requested from at least one of the user's terminal, the medical staff's terminal, and the external server.

Furthermore, the processor may further generate a request to transmit second exercise guide information for strengthening a heart condition of the user according to the heart rate analysis result to at least one of the user's terminal, the medical staffs terminal, and the external server which had generated the request when the command for transmitting the heart rate analysis result is requested, and further transmit the second exercise guide information to at least one of the user's terminal, the medical staffs terminal, and the external server which had generated the request when a command for transmitting the second exercise guide information is requested from at least one of the user's terminal, the medical staffs terminal, and the external server which had generated the request.

Furthermore, the processor may further receive body temperature-specific heart rate information of the user who uses the virtual reality device measured by a body temperature sensor and the heart rate sensor of the patch-type sensor, further determine whether a degree of body temperature-specific heart rate information of the user satisfies a preset body temperature-specific heart rate level for the preset time period as a result of further analyzing the body temperature-specific heart rate information through the artificial intelligence model, further store a body temperature-specific heart rate analysis result for the preset time period when the degree of body temperature-specific heart rate of the user does not satisfy the body temperature-specific heart rate level, further generate a request to transmit the body temperature-specific heart rate analysis result to at least one of the user's terminal, the medical staff's terminal related to the user's terminal, and the external server related to the user's terminal when storage of the body temperature-specific heart rate analysis result is completed, and transmit the body temperature-specific heart rate analysis result to at least one of the user's terminal, the medical staffs terminal, and the external server which had generated the request when a command for transmitting the body temperature-specific heart rate analysis result is requested from at least one of the user's terminal, the medical staffs terminal, and the external server.

Furthermore, the processor may further generate a request to transmit third exercise guide information for strengthening a body temperature-specific heart state of the user according to the temperature-specific heart rate analysis result to at least one of the user's terminal, the medical staffs terminal, and the external server which had generated the request when the command for transmitting the temperature-specific heart rate analysis result is requested, and further transmit the third exercise guide information to at least one of the user's terminal, the medical staffs terminal, and the external server which had generated the request when a command for transmitting the third exercise guide information is requested from at least one of the user's terminal, the medical staffs terminal, and the external server which had generated the request.

Furthermore, the virtual reality device may provide preset game-type content for each breathing exercise course to analyze the user's breathing exercise.

According to an embodiment, a server for providing health care services includes a user's terminal, a medical staffs terminal related to the user's terminal, an external server related to the user's terminal, a patch-type sensor, and a server that communicates with at least one of the user's terminal, the medical staffs terminal, the external server, and the patch-type sensor, wherein the server may receive a biological signal of a sleeping user measured by the patch-type sensor, determine whether a sleep state of the user satisfies a preset sleep state condition for a preset time period as an analysis result of analyzing the biological signal through an artificial intelligence model, store the analysis result for the preset time period when the sleep state of the user does not satisfy the sleep state condition, generate a request to transmit the analysis result to at least one of the user's terminal, the medical staffs terminal, and the external server when the storing of the analysis result is completed, and transmit the analysis result to at least one of the user's terminal, the medical staffs terminal, and the external server which has generated a request when a command for transmitting the analysis result is requested from at least one of the user's terminal, the medical staffs terminal, and the external server.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram illustrating a a patch-type sensor-based health management service providing system using artificial intelligence of the inventive concept;

FIG. 2 illustrates a configuration of the patch-type sensor of FIG. 1;

FIG. 3 illustrates a configuration of a server of FIG. 1;

FIGS. 4 and 5 are flowcharts illustrating a sleep management method based on a patch-type sensor using artificial intelligence, which is performed by the server of FIG. 1;

FIG. 6 is a diagram illustrating a process of outputting sleep state determination data according to the input of biometric data corresponding to bio-signals based on the artificial intelligence model of the server of FIG. 1 as an example;

FIGS. 7 to 9 are diagrams illustrating, as an example, a process of transmitting guide information according to the analysis result analyzed by the server of FIG. 1 to at least one of a user's terminal, a medical staffs terminal, an external server, and a guardian's terminal;

FIG. 10 illustrates a configuration of a virtual reality device of FIG. 1;

FIGS. 11 to 14 are diagrams illustrating game types of game-type content displayed through the display device of a virtual reality device of FIG. 10 as examples;

FIGS. 15 to 20 are flowcharts illustrating a method of providing a virtual reality-based breathing exercise service using a patch-type sensor, which is performed by the server of FIG. 1 as examples;

FIG. 21 illustrates a process of outputting breathing exercise state determination data, heart rate state determination data, and body temperature-specific heart rate state determination data according to the input of biometric data corresponding to bio signals based on the artificial intelligence model of the server of FIG. 1; and

FIGS. 22 to 24 are diagrams illustrating, as an example, a process of transmitting exercise guide information according to the analysis result analyzed by the server of FIG. 1 to at least one of a user's terminal, a medical staffs terminal, an external server, and a guardian's terminal.

### DETAILED DESCRIPTION

Like reference numerals refer to like elements throughout the specification. The inventive concept does not describe all elements of the embodiments, and general content or overlapping content between the embodiments in the technical field to which the inventive concept belongs is omitted. The term ‘unit, module, member, or block’ used in the specification may be implemented as software or hardware, and according to embodiments, a plurality of ‘units, modules, members, or blocks’ may be implemented as one component, one ‘part, module, member, block’ may include a plurality of components.

Throughout the specification, when a part is said to be “connected” to another part, this includes not only the case of being directly connected but also the case of being indirectly connected, and the indirect connection includes being connected through a wireless communication network.

In addition, unless explicitly described to the contrary, the word “comprise” and variations such as “comprises” or “comprising” will be understood to imply the inclusion of stated elements but not the exclusion of any other elements.

Throughout the specification, when a member is said to be located “on” another member, this includes not only a case where a member is in contact with another member, but also a case where another member exists between the two members.

Terms such as first and second are used to distinguish one component from another, and the components are not limited by the aforementioned terms.

As used herein, singular forms may include plural forms as well unless the context clearly indicates otherwise.

In each step, identification symbols are used for convenience of description and the identification symbols do not describe the order of the steps and the steps may be performed in a different order from the described order unless the context clearly indicates a specific order.

Hereinafter, the working principle and embodiments of the inventive concept will be described with reference to the accompanying drawings.

In this specification, a patch-type sensor-based sleep management service providing system using artificial intelligence according to the inventive concept includes various devices capable of providing results to users by performing calculation processing. For example, the patch-type sensor-based sleep management service providing system using artificial intelligence according to the inventive concept may include a computer, a server, and a portable terminal, or may have the form of any one of thereof.

Here, the computer may include, for example, a notebook, a desktop, a laptop, a tablet PC, a slate PC, and the like equipped with a web browser.

The server processes information by communicating with an external device, and may include an application server, a computing server, a database server, a file server, a mail server, a proxy server, and a web server.

The portable terminal is, for example, a wireless communication device that ensures portability and mobility, and may include all kinds of handheld-based wireless communication devices such as a Personal Communication System (PCS), Global System for Mobile communications (GSM), Personal Digital Cellular (PDC), Personal Handyphone System (PHS), PDA (Personal Handyphone System), PDA (Personal Digital Assistant), IMT (International Mobile Telecommunication)-2000, CDMA (Code Division Multiple Access)-2000, W-CDMA (W-Code Division Multiple Access), or WiBro (Wireless Broadband Internet) terminal or a smart phone, and wearable devices such as watches, rings, bracelets, anklets, necklaces, glasses, contact lenses, head-mounted-devices (HMDs).

A health care service providing system may receive a biological signal of a sleeping user measured by the patch-type sensor, determine whether a sleep state of the user satisfies a preset sleep state condition for a preset time period as an analysis result of analyzing the biological signal through an artificial intelligence model, store the analysis result for the preset time period when the sleep state of the user does not satisfy the sleep state condition, generate a request to transmit the analysis result to at least one of the user's terminal, the medical staffs terminal, and the external server when the storage of the analysis result is completed, and transmit the analysis result to at least one of the user's terminal, the medical staffs terminal, and the external server which has generated a request when a command for transmitting the analysis result is requested from at least one of the user's terminal, the medical staffs terminal, and the external server.

Hereinafter, the health care service providing system will be described in detail.

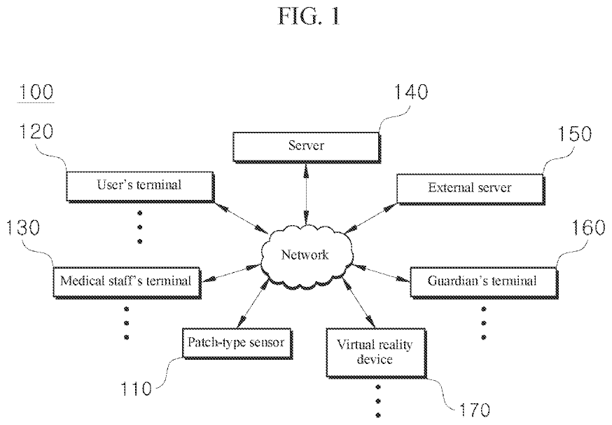

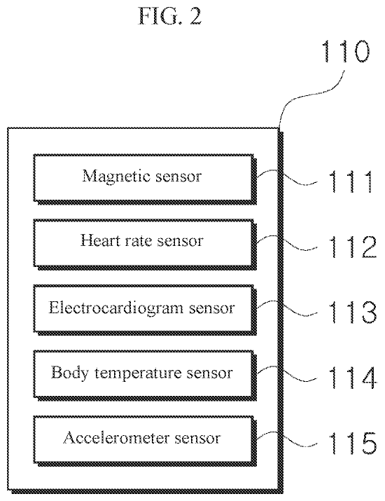

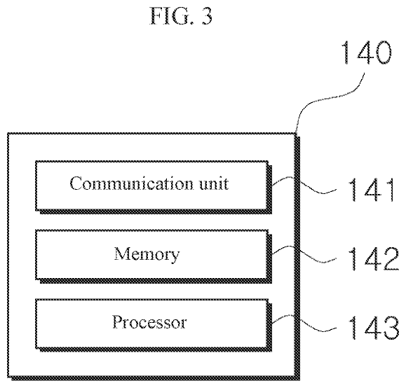

FIG. 1 is a diagram illustrating a patch-type sensor-based health management service providing system using artificial intelligence of the inventive concept. FIG. 2 illustrates a configuration of a patch-type sensor of FIG. 1. FIG. 3 illustrates a configuration of a server of FIG. 1.

Referring to FIGS. 1 to 3, a patch-type sensor-based health management service providing system 100 using artificial intelligence may include a patch-type sensor 110, a user's terminal 120, a medical staff's terminal 130, a server 140, and an external server 150.

The patch-type sensor 110 is a patch-type subminiature sensor and may be attached to a part of a user's body so as to measure the user's biosignal in real time. For example, the patch-type sensor 110 may be attached to the chest and abdomen near the user's heart to measure one or more bio-signals of the user, or may be attached to a part of the user's body, such as the user's wrist or the user's nose.

The patch-type sensor 110 may include a micro-sized battery and a module for WiFi or Bluetooth, or may include a module capable of communicating with an Android device and an Apple device including an iPhone and realizing an audiovisual effect by using the module for WiFi or Bluetooth. In addition, the patch-type sensor 110 may include a sleep mode and a hibernation mode for increasing a battery usage time, may include a battery usage display device for displaying a battery usage status, and may include a battery exchange device for displaying a battery replacement status.

As shown in FIG. 2, the patch-type sensor 110 may include multiple sensors such as a magnetic sensor 111, a heart rate sensor 112, an electrocardiogram sensor 113, a body temperature sensor 114, an acceleration sensor 115, and the like.

The magnetic sensor 111 may be provided to directly measure the breathing state of a sleeping user. Here, the magnetic sensor 111 may be a respiratory abnormality detection sensor that measures an abnormal state of respiration, and may transmit measured information related to sleep breathing of the user to the server 140. The magnetic sensor 111 may measure the magnetic field direction of a permanent magnet with respect to a plurality of different axial directions. In this case, the magnetic sensor 111 may measure a positional change through the density of magnetic force lines of the permanent magnet. For example, the magnetic sensor 111 may also include a MEMS sensor that includes a permanent magnet fixed to another part of the user's body to generate a magnetic field, processes calculation for a vector value obtained by detecting the density of magnetic force lines of the permanent magnet, and measure the calculated positional change. The MEMS sensor may transmit the measured positional change to the server 140 so as to be shown as a graph. In addition, the magnetic sensor 111 may set the maximum exhalation and maximum inhalation according to a subject's respiration and transmit the relative positional change to the server 140 so as to be shown as a graph.

The heart rate sensor 112 may be provided to directly measure the heart rate of a sleeping user. The electrocardiogram sensor 113 may be provided to directly measure the electrocardiogram of a sleeping user. The electrocardiogram sensor 113 may measure the current and potential of the heart. The heart rate sensor 112 and the electrocardiogram sensor 113 may have superior performance to conventional smart watches, because the heart rate sensor 112 and the electrocardiogram sensor 113 are provided at locations close to the heart. The heart rate sensor 112 may transmit information about the measured heart rate of the user to the server 140 so as to predict heart abnormalities and use the information as sleep analysis data. The electrocardiogram sensor 113 may transmit information about the measured electrocardiogram of the user to the server 140 so as to predict heart abnormalities and use the information as sleep analysis data.

The body temperature sensor 114 may be provided to directly measure the body temperature of a sleeping user. The acceleration sensor 115 may be provided to directly measure the movement of a sleeping user. The body temperature sensor 114 may transmit information about the measured body temperature of the user to the server 140 to be used as sleep analysis data, and the acceleration sensor 115 may transmit information about the measured movement of the user to the server 140 to be used as sleep analysis data.

Because a sensor for measuring a bio-signal is manufactured in the form of a patch, the patch-type sensor 110 may minimize the user's discomfort during sleep, thus enabling analysis of natural bio-responses. On the other hand, the patch-type sensor 110 may further include a vibrator that notifies when the period or position value set by the user is out of a reference range.

The server 140 may include a communication unit 141, a memory 142, and a processor 143.

The communication unit 141 may communicate with at least one of the patch-type sensor 110, the user's terminal 120, the terminal 130 of a medical staff related to the user's terminal 120, and the external server 150 related to the user's terminal 120. Also, the communication unit 141 may communicate with the terminal 160 of a guardian related to the terminal 120 of the user. In this case, the user's terminal 120 may be a sleeping subject's terminal or a patient's terminal, the medical staffs terminal 130 may be a doctor's terminal managing a sleeping subject or patient, and the external server 150 may be a server for externally managing information about a sleeping subject or patient's sleeping condition. For example, the external server 150 may be at least one of a hospital server, and a company server and a research center server for researching sleep conditions. The guardian's terminal 160 may be a terminal that protects a sleeping subject or patient.

The communication unit 141 may include not only a WiFi module and a wireless broadband module, but also wireless communication modules supporting various wireless communication schemes such as global system for mobile communication (GSM), code division multiple access (CDMA), wideband code division multiple access (WCDMA), universal system mobile telecommunications system (UMTS), time division multiple access (TDMA), long term evolution (LTE), 4G, 5G, and 6G.

The memory 142 may store data for algorithms for controlling the operations of the components in the present device or programs reproducing the algorithms, and at least one processor 143 may be implemented for performing the above-described operations using the data stored in the memory 142. Here, the memory 142 and the processor 143 may be implemented as separate chips. Also, the memory 142 and the processor 143 may be implemented as a single chip.

The memory 142 may store data supporting various functions of the present device, programs for operation of the processor 143, input/output data, a plurality of application programs or applications running in the present device, data and commands for operation of the present device. At least some of these application programs may be downloaded from an external server through wireless communication.

The memory 142 may include at least one type of storage medium among a flash memory type, a hard disk type, a solid state disk type, an Silicon Disk Drive type (SDD) type, a multimedia card micro type, a card type memory (e.g., SD or XD memory), random access memory (RAM), static random access memory (SRAM), read-only memory (ROM), electrically erasable programmable read-only memory (EEPROM) programmable read-only memory (PROM), magnetic memory, magnetic disk, and optical disk. In addition, the memory 142 may be a database that is separated from the present device, but connected by wire or wirelessly.

The processor 143 may control operations related to provision of a patch-type sensor-based health management service using artificial intelligence.

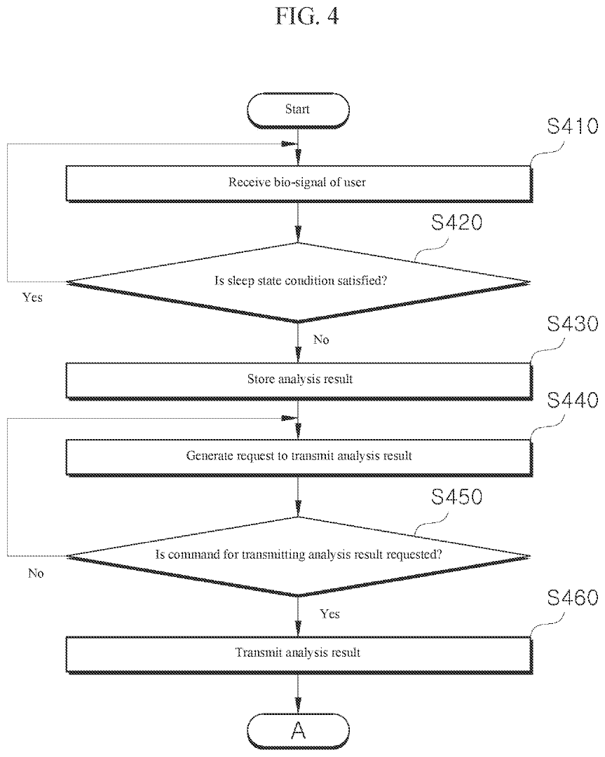

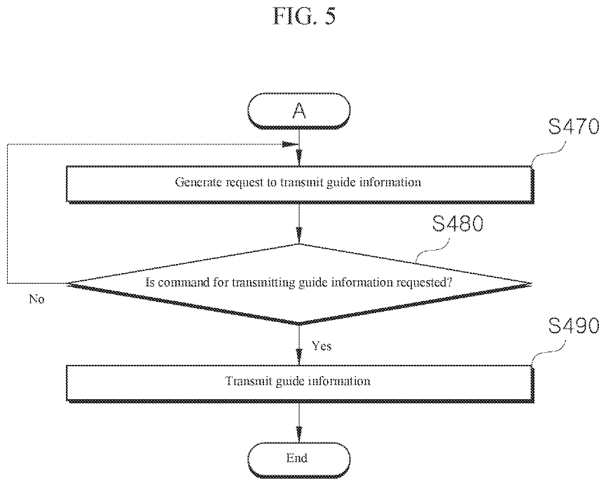

FIGS. 4 and 5 are flowcharts illustrating a sleep management method based on a patch-type sensor using artificial intelligence, which is performed by the server of FIG. 1.

Referring to FIGS. 4 and 5, the server 140 may receive the biological signal of a sleeping user, which is measured by the patch-type sensor 110 (S410).

In this case, the server 140 may receive information related to sleep breathing of a user measured through the magnetic sensor 111 of the patch-type sensor 110. The server 140 may receive information related to sleep breathing measured based on a change in magnetic force of the magnetic sensor 111 attached to the chest. In addition, the server 140 may receive information about the user's heart rate measured through the heart rate sensor 112 of the patch-type sensor 110, and receive information about the user's electrocardiogram measured through the electrocardiogram sensor 113 of the patch-type sensor 110. In addition, the server 140 may receive information about the user's body temperature measured through the body temperature sensor 114 of the patch-type sensor 110, and receive information about the movement of the user measured through the acceleration sensor 115 of the patch-type sensor 110.

The server 140 may determine whether the sleep state of the user satisfies a preset sleeping condition for a preset time period, as a result of analyzing the biological signals of the sleeping user through an artificial intelligence model.

In this case, the server 140 may determine whether the level of breathing of the user satisfies a preset breathing level for a preset time period based on the received sleep breathing-related information. In addition, the server 140 may determine whether the level of the heart rate of the user satisfies a preset heart rate level for a preset time period based on the received heart rate information. Also, the server 140 may determine whether the level of the ECG of the user satisfies a preset ECG level for a preset time period, based on the received ECG information. In addition, the server 140 may determine whether the level of the body temperature of the user satisfies a preset body temperature level for a preset time period. In addition, the server 140 may determine whether the level of the sleeping posture of the user satisfies a preset sleeping posture level for a preset time period.

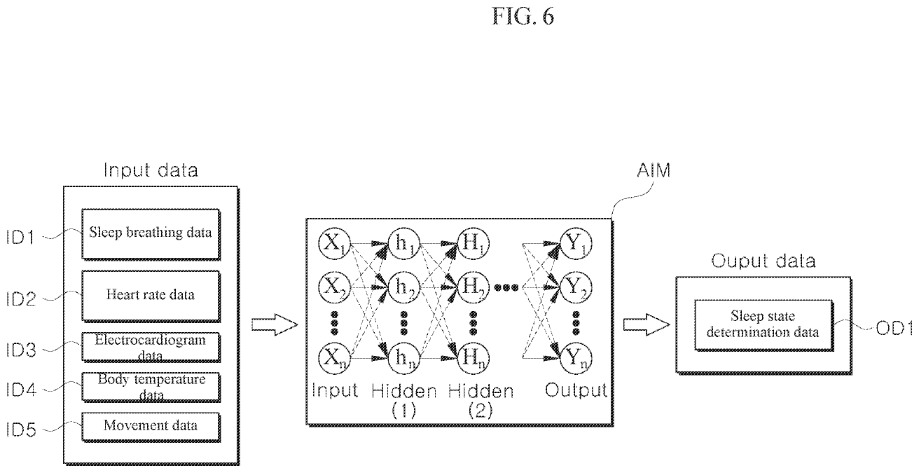

FIG. 6 is a diagram illustrating a process of outputting sleep state determination data according to the input of biometric data corresponding to bio-signals based on the artificial intelligence model of the server of FIG. 1 as an example.

Referring to FIG. 6, the server 140 may input, into an artificial intelligence model (AIM), sleep breathing related data ID1 received from the magnetic sensor 111, heart rate data ID2 received from the heart rate sensor 112, electrocardiogram data ID3 received from the electrocardiogram sensor 113, body temperature data ID4 received from the body temperature sensor 114, and movement data ID5 received from the acceleration sensor 115, which are all metadata, as input values, and output a result value of sleep state determination data OD1 determined by performing learning based on the artificial intelligence model (AIM). Here, the sleep state determination data OD1 may be data for determining whether or not a sleep state condition is satisfied or unsatisfied according to the input of the sleep breathing related data ID1, the heart rate data ID2, the electrocardiogram data ID3, the body temperature data ID4, and the movement data ID5.

The artificial intelligence model (AIM) may be built to learn the sleep breathing related data ID1, the heart rate data ID2, the electrocardiogram data ID3, the body temperature data ID4, and the movement data ID5 included in the input data, through correlation. The artificial intelligence model (AIM) may construct the sleep breathing related data ID1, the heart rate data ID2, the electrocardiogram data ID3, the body temperature data ID4, and the movement data ID5 as a training dataset by using the CNN algorithm or RNN algorithm and may be trained in a reinforced scheme.

The server 140 may output data indicating whether conditions of the sleep state determination data OD1 are satisfied or unsatisfied according to the input of the sleep breathing-related data, the heart rate data, the electrocardiogram data, the body temperature data, and the movement data based on the artificial intelligence model (AIM).

When the server 140 outputs the sleep state determination data OD1, the server 140 may output first sleep respiration-related data in which conditions of the sleep state determination data OD1 are unsatisfied and also second sleep respiration-related data which is improved to satisfy the conditions. In addition, when the server 140 outputs the sleep state determination data OD1, the server 140 may output first heart rate data in which conditions of the sleep state determination data OD1 are unsatisfied and also second heart rate data which is improved to satisfy the conditions. In addition, when outputting the sleep state determination data OD1, the server 140 may output first ECG data in which conditions of the sleep state determination data OD1 are unsatisfied and also second ECG data which is improved to satisfy the conditions. In addition, when outputting the sleep state determination data OD1, the server 140 may output first body temperature data in which conditions of the sleep state determination data OD1 are unsatisfied and also second body temperature data which is improved to satisfy the conditions. Also, when outputting the sleep state determination data OD1, the server 140 may output first movement data in which conditions of the sleep state determination data OD1 are unsatisfied and also second movement data which is improved to satisfy the conditions.

When the sleep state of the user does not satisfy the sleeping state conditions (S420), the server 140 may store an analyzed result for a predetermined time (S430). In this case, the server 140 may store an analyzed first result for a predetermined time when the level of the breathing of the user does not satisfy a preset respiration level. In addition, the server 140 may store an analyzed second result for a predetermined time when the level of the heart rate of the user does not satisfy the preset heart rate level. In addition, the server 140 may store an analyzed third result for a predetermined time when the level of the ECG of the user does not satisfy the preset ECG level. In addition, the server 140 may store an analyzed fourth result for a predetermined time when the level of the body temperature of the user does not satisfy the preset body temperature level. In addition, the server 140 may store an analyzed fifth result for a predetermined time when the level of the sleeping posture of the user does not satisfy the preset sleeping posture level.

When the storage of the analyzed results have been completed, the server 140 may generate a request to transmit the analysis results to at least one of the user's terminal 120, the medical staffs terminal 130, and the external server 150 (S440). In addition, when the storage of the analyzed results have been completed, the server 140 may generate a request to transmit the analysis results to the terminal 160 of the guardian who protects the user's terminal 120. That is, when the storage of at least one of the analyzed first to fifth results has been completed, the server 140 may generate a request to transmit at least one of the first to fifth analysis results to at least one of the user's terminal 120, the medical staffs terminal 130, the external server 150, and the guardian's terminal 160.

When a command for transmitting the analysis results is requested from at least one of the user's terminal 120, the medical staffs terminal 130, and the external server 150 (S450), the server 140 may transmit the analysis result to at least one of the user's terminal 120, the medical staffs terminal 130 and the external server 150, which has generated a request (S460). In addition, when a command for transmitting the analysis results is requested from the terminal 160 of the guardian protecting the user's terminal 120, the server 140 may transmit the analysis results to the terminal 160 of the guardian who has requested the analysis results. That is, when a command for transmitting at least one of the first to fifth analysis results is requested from at least one of the user's terminal 120, the medical staffs terminal 130, and the external server 150, the server 140 may transmit at least one of the first to fifth analysis results to at least one of the user's terminal 120, the medical staff's terminal 130, the external server 150, and the guardian's terminal 160, which has requested at least one of the first analysis result to the fifth analysis result. In this case, the server 140 may transmit at least one of the first analysis result to the fifth analysis result stored for a predetermined time to at least one of the user's terminal 120, the medical staffs terminal 130, the external server 150, and the guardian's terminal 160.

In addition, the server 140 may notify at least one of the user's terminal 120, the medical staffs terminal 130, the external server 150, and the guardian's terminal 160, which has generated a request, of a warning situation through a notification means such as a pop-up window, text message, or SNS message, when, as a result of the first analysis, the level of the breathing of the user does not satisfy the preset breathing level for a preset time period, for example, the user's apneic period becomes too long; when, as a result of the second analysis, the heart rate of the user does not satisfy the preset heart rate level for a preset time period, for example, a change in the heart rate of the user is too high; when, as a result of the third analysis, the level of the electrocardiogram of the user does not satisfy the preset electrocardiogram level for a preset time period, for example, a change in the electrocardiogram of the user is too high; when, as a result of the fourth analysis, the level of the body temperature of the user does not satisfy the preset body temperature level for a preset time period, for example, the body temperature of the user is high or the body temperature changes abruptly; or when, as a result of the fifth analysis, the degree of the sleeping posture of the user does not satisfy the preset sleeping posture level for a preset time period, for example, the user's sleeping posture is severe enough to strain the body.

The server 140 may transmit a result of analysis based on the sleep state determination data OD1 output by the artificial intelligence model (AIM) according to the input of sleep breathing data, heart rate data, electrocardiogram data, body temperature data, and movement data to at least one of the user's terminal 120, the medical staffs terminal 130, the external server 150, and the guardian's terminal 160, which has generated a request.

The server 140 may transmit an accurate analysis result of the sleep state for each user to at least one of the user's terminal 120, the medical staffs terminal 130, the external server 150, and the guardian's terminal 160, thus providing a sleep state analysis service for each user.

When a command for transmitting the analysis results is requested from at least one of the user's terminal 120, the medical staffs terminal 130, and the external server 150 (S450), the server 140 may generate a request to transmit guide information for improving the sleep state of the user according to the analysis result to at least one of the user's terminal 120, the medical staffs terminal 130, and the external server 150, which has generated a request (S470). In addition, when a command for transmitting the analysis results is requested from the terminal 160 of the guardian protecting the user's terminal 120, the server 140 may generate a request to transmit guide information for improving the sleep state of the user according to the analysis result of the guardian's terminal 160 which has requested the analysis result. That is, when a command for transmitting at least one of the first to fifth analysis results is requested from at least one of the user's terminal 120, the medical staffs terminal 130, and the external server 150, the server 140 may generate a request to transmit the guide information for improving the sleep state of the user according to at least one of the first analysis result to the fifth analysis result to at least one of the user's terminal 120, the medical staffs terminal 130, the external server 150, and the guardian's terminal 160, which has requested at least one of the first analysis result to the fifth analysis result.

When a command for transmitting the guide information is requested from at least one of the user's terminal 120, the medical staffs terminal 130, and the external server 150, which has generated a request (S480), the server 140 may further transmit the guide information to at least one of the user's terminal 120, the medical staffs terminal 130, and the external server 150, which has requested guide information (S490). In this case, when the command for transmitting guide information is requested from the terminal 160 of the guardian which has generated a request, the server 140 may further transmit the guide information to the terminal 160 of the guardian which has requested the guide information. In this case, the server 140 may transmit at least one of first guide information to fifth guide information for improving the sleep state of the user according to at least one of the first analysis result to the fifth analysis result stored for a predetermined time to at least one of the user's terminal 120, the medical staffs terminal 130, the external server 150, and the guardian's terminal 160. That is, the server 140 may transmit at least one of first guide information according to the first analysis result, second guide information according to the second analysis result, third guide information according to the third analysis result, fourth guide information according to the fourth analysis result and fifth guide information according to the fifth analysis result to at least one of the user's terminal 120, the medical staffs terminal 130, the external server 150, and the guardian's terminal 160.

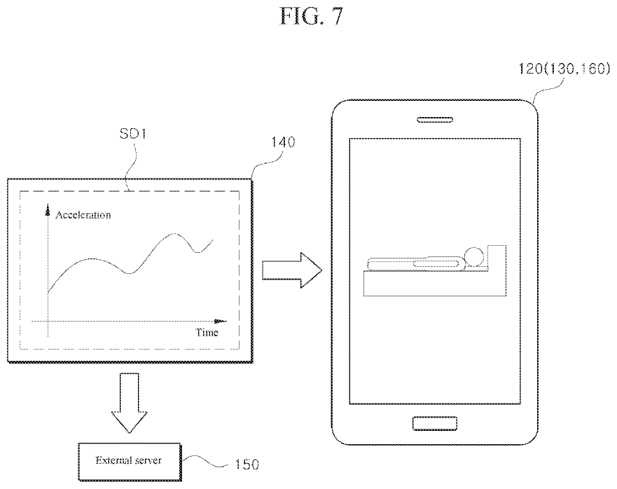

FIGS. 7 to 9 are diagrams illustrating, as an example, a process of transmitting guide information according to the analysis result analyzed by the server of FIG. 1 to at least one of a user's terminal, a medical staffs terminal, an external server, and a guardian's terminal.

Referring to FIG. 7, the server 140 may transmit the fifth guide information according to fifth analysis result information SD1 obtained by analyzing the position of the sleeping posture of the user to at least one of the user's terminal 120, the medical staffs terminal 130, the external server 150, and the guardian's terminal 160. In this case, the fifth analysis result information SD1 may be an change value in acceleration obtained by analyzing the user's movement while sleeping based on the acceleration sensor 115. In this case, the server 140 may transmit the fifth guide information for improving the movement in the sleeping state based on the fifth analysis result information SD1 to at least one of the user's terminal 120, the medical staffs terminal 130, the external server 150, and the guardian's terminal 160. For example, the fifth guide information may include an image of movement in a sleeping state acquired by an external camera, and information providing a corrected posture of the user before going to bed to minimize movement in the user's sleeping state. In this case, at least one of the user's terminal 120, the medical staffs terminal 130, and the guardian's terminal 160 may display the fifth guide information corresponding to an image of movement in a sleeping state on a screen, and the external server 150 may receive and database at least one of the fifth analysis result information SD1 and the fifth guide information. At least one of researchers and medical staffs may perform at least one of research and treatment on movement in a sleeping state by using at least one of the fifth analysis result information SD1 and the fifth guide information, which has databased.

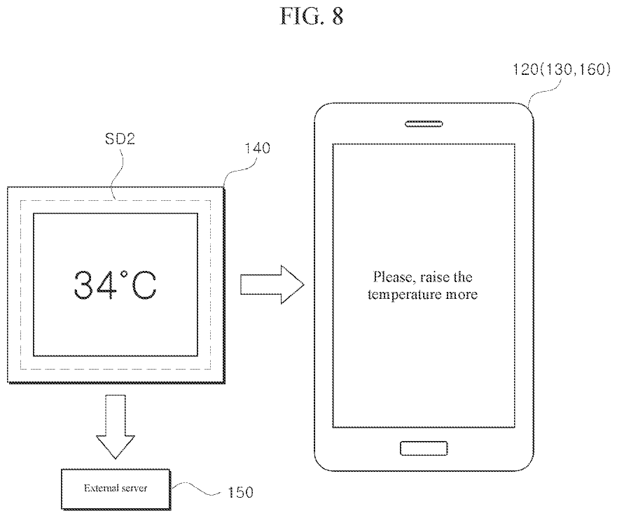

Referring to FIG. 8, the server 140 may transmit the fourth guide information according to fourth analysis result information SD2 obtained by analyzing the body temperature of the user to at least one of the user's terminal 120, the medical staffs terminal 130, the external server 150, and the guardian's terminal 160. In this case, the server 140 may determine hypothermia when the user's body temperature is lower than a preset reference value based on the body temperature of the user, and transmit fourth guide information for guiding the user to set a sleeping temperature to at least one of the user's terminal 120, the medical staffs terminal 130, the external server 150, and the guardian's terminal 160. In this case, at least one of the user's terminal 120, the medical staffs terminal 130, and the guardian's terminal 160 may display the fourth guide information on the screen. For example, at least one of the user's terminal 120, the medical staffs terminal 130, and the guardian's terminal 160 may display the fourth guide information “Please increase the temperature!” on the screen. In this case, the external server 150 may receive and database at least one of the fourth analysis result information SD2 and the fourth guide information and at least one of researchers and medical staffs may perform at least one of research and treatment on body temperature in a sleeping state by using at least one of the fourth analysis result information SD2 and the fourth guide information, which has databased.

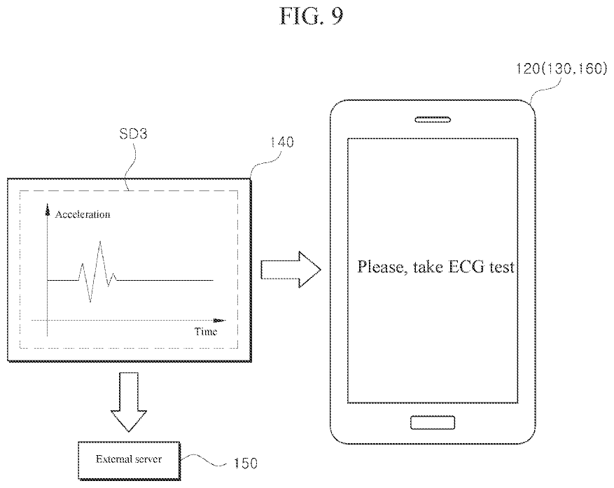

Referring to FIG. 9, the server 140 may transmit the third guide information according to third analysis result information SD3 obtained by analyzing the ECG of the user to at least one of the user's terminal 120, the medical staffs terminal 130, the external server 150, and the guardian's terminal 160. In this case, the server 140 may determines that the user's electrocardiogram is not normal when a change value in the user's electrocardiogram does not satisfy a predetermined reference value based on the change value in the user's electrocardiogram, and transmit the third guide information for proposing an electrocardiogram test to at least one of the user's terminal 120, the medical staffs terminal 130, the external server 150, and the guardian's terminal 160. In this case, at least one of the user's terminal 120, the medical staffs terminal 130, and the guardian's terminal 160 may display the third guide information on the screen. For example, at least one of the user's terminal 120, the medical staffs terminal 130, and the guardian's terminal 160 may display the third guide information “Please, take an electrocardiogram test! !” on the screen. In this case, the external server 150 may receive and database at least one of the third analysis result information SD3 and the third guide information and at least one of researchers and medical staffs may perform at least one of research and treatment on ECG in a sleeping state by using at least one of the third analysis result information SD3 and the third guide information, which has databased.

On the other hand, although not shown, the server 140 may transmit the first guide information according to the first analysis result obtained by analyzing the level of the user's breathing and the second guide information according to the second analysis result obtained by analyzing the level of the user's heart rate, to at least one of the user's terminal 120, the medical staffs terminal 130, the external server 150, and the guardian's terminal 160. In this case, at least one of the user's terminal 120, the medical staffs terminal 130, and the guardian's terminal 160 may display the first guide information and the second guide information on the screen. In this case, the external server 150 may receive and database at least one of the first analysis result information, the first guide information, the second analysis result information, and the second guide information, and at least one of researchers and medical staffs may perform at least one of research and treatment on sleep apnea and heart rate in a sleeping state by using at least one of the first analysis result information, the first guide information, the second analysis result information, and the second guide information, which has databased.

The server 140 may transmit a result obtained by performing analysis based on the sleep state determination data OD1 output by the artificial intelligence model (AIM) according to the input of sleep breathing data, heart rate data, electrocardiogram data, body temperature data, and movement data, and also guide information for improving the user's sleep state according to the analysis result, to at least one of the user's terminal 120, the medical staffs terminal 130, the external server 150, and the guardian's terminal 160, which has generated a request.

The server 140 may transmit an accurate analysis result of the sleep state and guide information for improving the sleep state for each user to at least one of the user's terminal 120, the medical staffs terminal 130, the external server 150, and the guardian's terminal 160, thus providing a sleep state analysis service and an improvement service for each user in a customized manner.

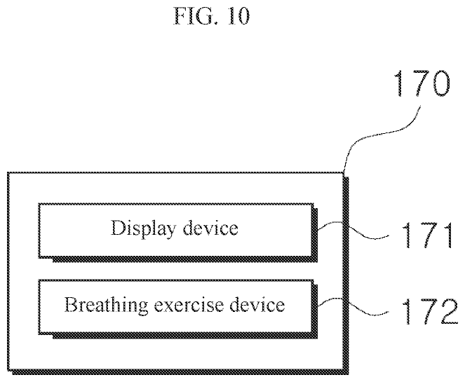

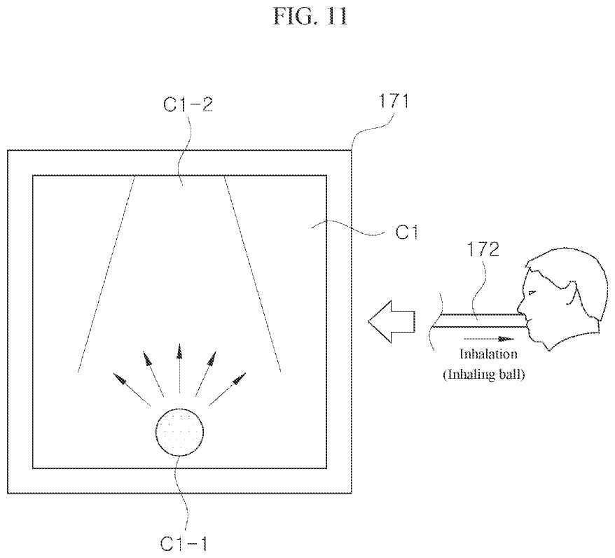

FIG. 10 illustrates a configuration of a virtual reality device of FIG. 1. FIGS. 11 to 14 are diagrams illustrating game types of game-type content displayed through the display device of a virtual reality device of FIG. 10 as examples.

A virtual reality device 170 may include a display device 171 and a breathing exercise device 172. The display device 171 may display game-type content predetermined for each breathing exercise course to analyze a user's breathing exercise. The breathing exercise device 172 may be provided to perform breathing exercise of a user according to the game type of the game-type content displayed through the display device 171.

For example, as shown in FIG. 11, the display device 171 may display a first game-like content C1 of a first breathing exercise course in which the user inhales a virtual reality-based ball C1-1 to analyze the user's breathing exercise. In this case, the virtual reality-based breathing exercise device C1-2 may implement a degree of inhalation of the ball C1-1 through the breathing exercise device 172 in cooperation with the breathing exercise device 172, and provide a training for increasing the amount of inhalation through a first breathing exercise course of inhaling the ball C1-1. In this case, the magnetic sensor 111 may detect the movement of the lungs according to the degree of inhaling the ball C1-1, and the processor 143 may calculate the amount of inhalation based on information about the movement of the lungs received from the magnetic sensor 111.

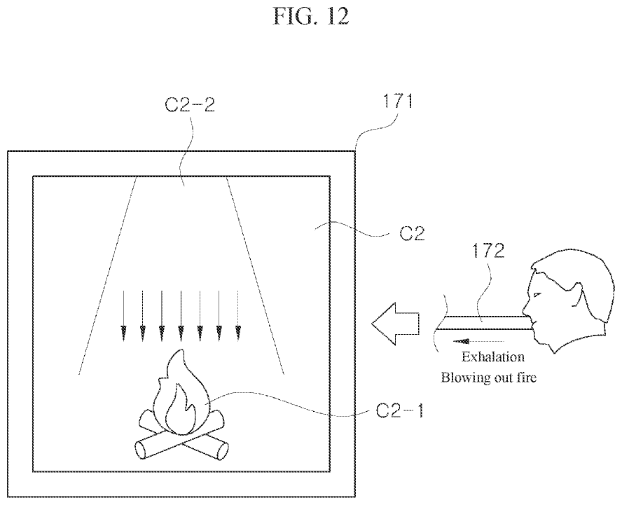

For another example, as shown in FIG. 12, the display device 171 may display a second game-like content C2 of a second breathing exercise course in which the user blows out a virtual reality-based fire C2-1 to analyze the user's breathing exercise. In this case, a virtual reality-based breathing exercise device C2-2 may implement a degree of exhalation of the ball C2-1 through the breathing exercise device 172 in cooperation with the breathing exercise device 172, and provide a training for increasing the amount of exhalation through a second breathing exercise course of blowing out the fire C2-1. In this case, the magnetic sensor 111 may detect the movement of the lungs according to the degree to which the user blows out the fire C2-1, and the processor 143 may calculate the amount of exhalation based on information about the movement of the lungs received from the magnetic sensor 111.

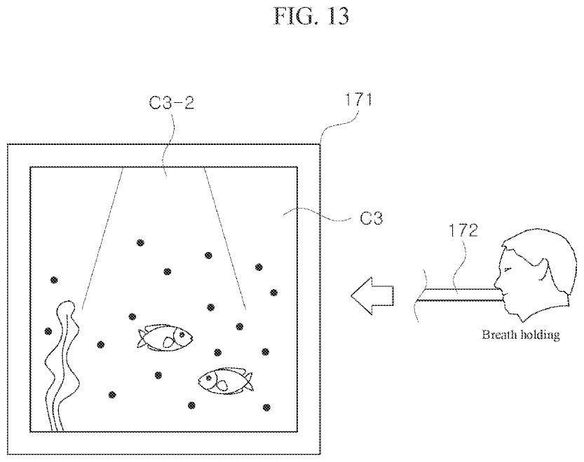

For another example, as shown in FIG. 13, the display device 171 may display a virtual reality-based third game-like content C3 of a third breathing exercise course in which a user holds the user's breath to analyze the user's breathing exercise, In this case, a virtual reality-based breathing exercise device C3-2 may implement a degree of breath-holding through the breathing exercise device 172 in cooperation with the breathing exercise device 172, and provide a training for increasing a lung capacity through the third breathing exercise course in which a user holds the user's breath. In this case, the magnetic sensor 111 may detect the movement of the lungs according to the degree of breath-holding, and the processor 143 may calculate the lung capacity based on information about the movement of the lungs received from the magnetic sensor 111.

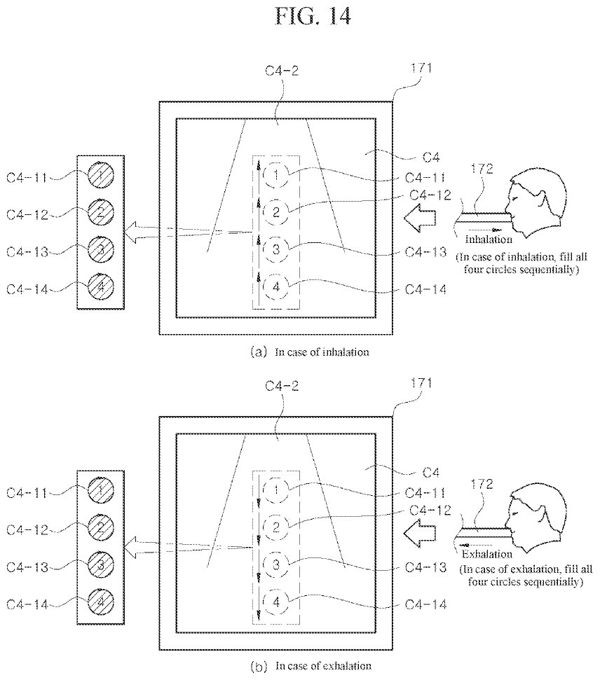

For another example, as shown in FIG. 14, the display device 171 may display a virtual reality-based fourth game-like content C4 of a fourth breathing exercise course in which a user breathes slowly to fill all four circles C4-11 to C4-14, to analyze the user's breathing exercise. In this case, a virtual reality-based breathing exercise device C4-2 may implement a degree to which four circles C4-11 to C4-14 are to be filled sequentially, according to the degree to which a user takes inhalation or the degree to which a user takes exhalation, through the breathing exercise device 172 in cooperation with the breathing exercise device 172, and provide a training for increasing the amount of inhalation, (a) of FIG. 14, and the amount of exhalation, (b) of FIG. 14, through the fourth breathing exercise course in which a user fills in all four circles C4-11 to C4-14. In this case, a method by which the four circles C4-11 to C4-14 are filled may be implemented in such a way that the first circle C4-11 in dotted line has rotated by 360-degrees while being gradually converted to solid line through inhalation or exhalation and is then filled, and the second circle C4-12, the third circle C4-13, and the fourth circle C4-14 are sequentially filled in the same way as above. In this case, the magnetic sensor 111 may detect the movement of the lungs according to the degree to which the user fills all four circles C4-11 to C4-14 by slowly breathing through inhalation or exhalation, and the processor 143 may calculate the amount of inhalation and the amount of exhalation based on information about the movement of the lungs received from the magnetic sensor 111.

The communication device unit 141 may communicate with at least one of the patch-type sensor 110, the user's terminal 120, the terminal 130 of a medical staff related to the user's terminal 120, the external server 150 related to the user's terminal 120, the device 160 of a guardian related to the user's terminal 120, and the virtual reality device 170. In this case, the user's terminal 120 may be a terminal of a subject who has completed breathing exercise or a patient's terminal, the medical staffs terminal 130 may be a terminal of a doctor managing the subject who has completed breathing exercise or the patient, and the external server 150 may be a server for externally managing information about a breathing exercise state of the subject who has completed breathing exercise or the patient For example, the external server 150 may be at least one of a hospital server, and a company server and a research center server for researching breathing exercise states. The guardian's terminal 160 may be a terminal that protects the subject who has completed breathing exercise or the patient.

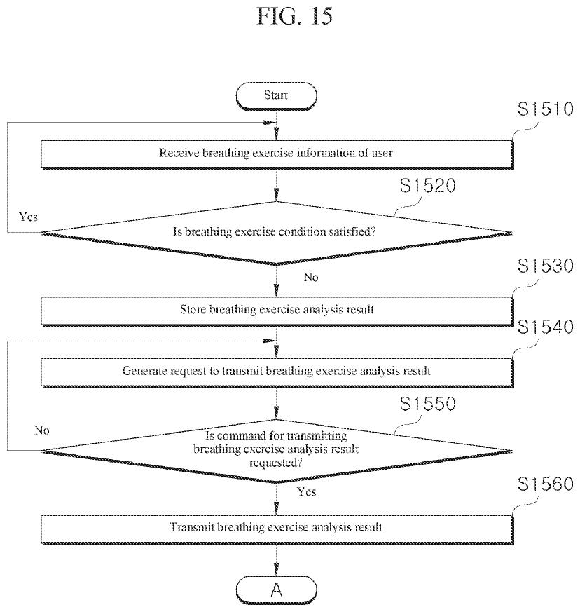

FIGS. 15 to 20 are flowcharts illustrating a method of providing a virtual reality-based breathing exercise service using a patch-type sensor, which is performed by the server of FIG. 1 as examples.

Referring to FIG. 15, the server 140 may receive breathing exercise information of a user using the virtual reality device 170 measured by the magnetic sensor 111 of the patch-type sensor 110 (S1510). As a result of analyzing the user's breathing exercise information through an artificial intelligence model, the server 140 may determine whether the degree of the user's breathing exercise satisfies a preset breathing exercise condition for a preset time period (S1520). In this case, the server 140 may receive breathing exercise information measured based on a change in magnetic force of the magnetic sensor 111 attached to the chest.

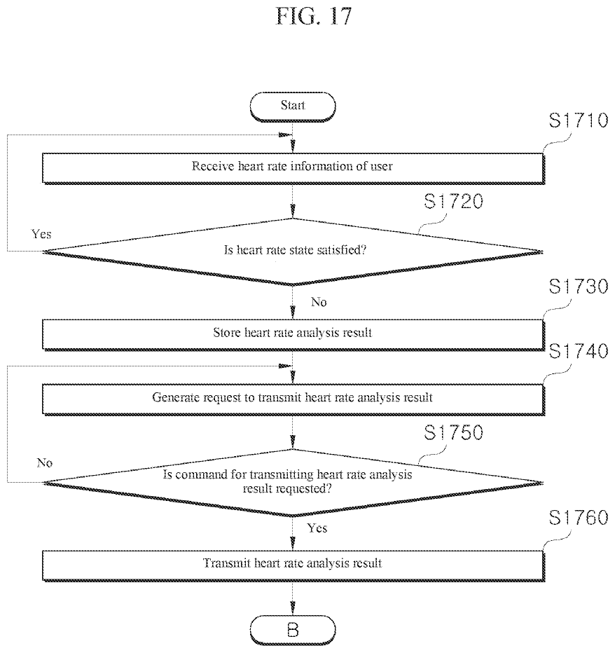

Referring to FIG. 17, the server 140 may further receive heart rate information of the user using the virtual reality device 170 which is measured by the heart rate sensor 112 of the patch-type sensor 110 (S1710). As a result of analyzing the heart rate information of the user through an artificial intelligence model, the server 140 may further determine whether the level of the user's breathing exercise satisfies a preset heart rate level for a preset time period (S1720).

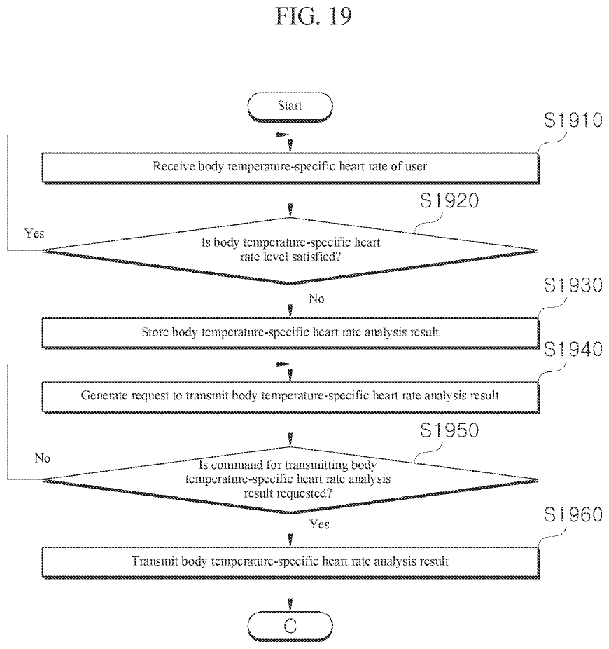

Referring to FIG. 19, the server 140 may further receive body temperature-specific heart rate information of the user using the virtual reality device 170 measured by the body temperature sensor 114 and the heart rate sensor 112 of the patch-type sensor 110 (S1910). As a result of further analyzing the user's body temperature-specific heart rate information through the artificial intelligence model, the server 140 may further determine whether the level of the user's body temperature-specific heart rate satisfies a preset body temperature-specific heart rate level for a preset time period (S1920).

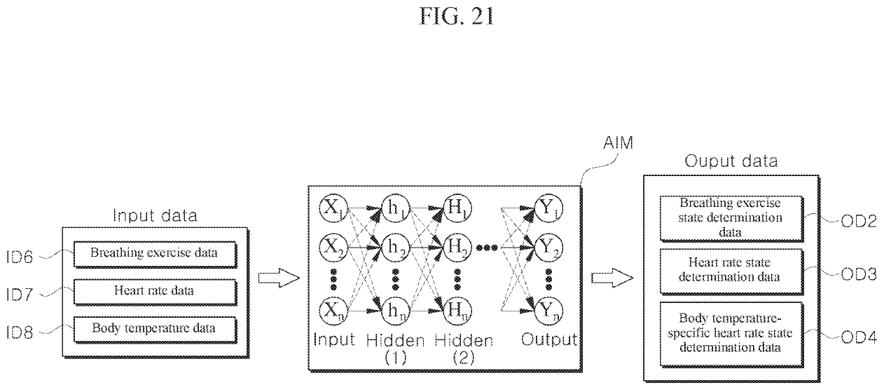

FIG. 21 illustrates a process of outputting breathing exercise state determination data, heart rate state determination data, and body temperature-specific heart rate state determination data according to the input of biometric data corresponding to bio signals based on the artificial intelligence model of the server of FIG. 1.

Referring to FIG. 21, the server 140 may input, into the artificial intelligence model (AIM), the input values of breathing exercise data ID6 received from the magnetic sensor 111, heart rate data ID7 received from the heart rate sensor 112, and body temperature data ID8 received from the body temperature sensor 114, which are all metadata, and output result values of breathing exercise state determination data OD2, heart rate state determination data OD3 according to the breathing exercise, and body temperature-specific heart rate state determination data OD4 according to the breathing exercise, which are all determined by learning based on the artificial intelligence model (AIM).

In this case, the breathing exercise state determination data OD2 may be data for determining whether the breathing exercise condition is satisfied or unsatisfied according to the input of the breathing exercise data ID6. In addition, the heart rate state determination data OD3 according to breathing exercise may be data for determining whether the heart rate level is satisfied or unsatisfied according to the input of the breathing exercise data ID6 and the heart rate data ID7. In addition, the body temperature-specific heart rate state determination data OD4 according to breathing exercise may be data for determining whether the body temperature-specific heart rate level is satisfied or unsatisfied according to the input of the breathing exercise data ID6, the heart rate data ID7 and the body temperature data ID8.

The artificial intelligence model (AIM) may be built to learn breathing exercise data ID6, the heart rate data ID7, and the body temperature data ID8 included in the input data, through correlation thereof. The artificial intelligence model (AIM) may construct learning data sets of the breathing exercise data ID6, the heart rate data ID7, and the body temperature data ID8 using a CNN algorithm or an RNN algorithm and may be trained in an reinforced manner.

The server 140 may output data indicating whether conditions of the breathing exercise state determination data OD2 are satisfied or unsatisfied according to the input of the breathing exercise data, the heart rate data, and the body temperature data based on the artificial intelligence model (AIM). In addition, when the server 140 outputs the breathing exercise state determination data OD2, the server 140 may output first breathing exercise data in which conditions of the breathing exercise state determination data OD2 are unsatisfied and also second breathing exercise data which is improved to satisfy the conditions. In addition, when the server 140 outputs the heart rate state determination data OD3 according to breathing exercise, the server 140 may output first heart rate data in which conditions of the heart rate state determination data OD3 are unsatisfied and also second heart rate data which is improved to satisfy the conditions. In addition, when the server 140 outputs the body temperature-specific heart rate state determination data OD3 according to breathing exercise, the server 140 may output first body temperature-specific heart rate data in which conditions of the heart rate state determination data OD3 are unsatisfied and also second body temperature-specific heart rate data which is improved to satisfy the conditions.

As shown in FIG. 15, when the level of the breathing exercise of the user does not satisfy the conditions of breathing exercise (S1520), the server 140 may store a breathing exercise analysis result for a predetermined time (S1530).

When the storage of the breathing exercise analysis result is completed, the server 140 may generate a request to transmit the breathing exercise analysis result to at least one of the user's terminal 120, the medical staff's terminal 130, and the external server 150 (S1540). In addition, when the storage of the breathing exercise analysis result is completed, the server 140 may generate a request to transmit the breathing exercise analysis result to the terminal 160 of a guardian protecting the user's terminal 120.

When a command for transmitting the breathing exercise analysis results is requested from at least one of the user's terminal 120, the medical staffs terminal 130, and the external server 150 (S1550), the server 140 may transmit the breathing exercise analysis result to at least one of the user's terminal 120, the medical staffs terminal 130, and the external server 150, which has generated a request (S1560). In addition, when a command for transmitting the breathing exercise analysis results is requested from the terminal 160 of the guardian, the server 140 may transmit the breathing exercise analysis result to the terminal 160 of the guardian who has requested the breathing exercise analysis result.

Furthermore, when, as a result of the breathing exercise analysis, the degree of breathing of the user does not satisfy a preset breathing level for the preset time period, for example, the user's respiratory volume is weak, the server 140 may notify at least one of the user's terminal 120, the medical staffs terminal 130, the external server 150, and the guardian's terminal 160, which has generated a request, of the warning situation via a notification such as a pop-up window, a text message, an SNS message, or the like.

As shown in FIG. 17, when the level of the user's heart rate does not satisfy a preset heart rate level (S1720), the server 140 may further store the heart rate analysis result for a predetermined time (S1730).

When the storage of the heart rate analysis result is completed, the server 140 may further generate a request to transmit the heart rate analysis result to at least one of the user's terminal 120, the medical staff's terminal 130, and the external server 150 (S1740). In addition, when the storage of the heart rate analysis result is completed, the server 140 may generate a request to transmit the heart rate analysis result to the terminal 160 of a guardian protecting the user's terminal 120.

When a command for transmitting the heart rate analysis result is requested from at least one of the user's terminal 120, the medical staffs terminal 130, and the external server 150 (S1750), the server 140 may transmit the heart rate analysis result to at least one of the user's terminal 120, the medical staffs terminal 130, and the external server 150, which has generated a request (S1760). In addition, when a command for transmitting the heart rate analysis result is requested from the terminal 160 of the guardian, the server 140 may transmit the heart rate analysis result to the terminal 160 of the guardian who has requested the heart rate analysis result.

Furthermore, when, as the heart rate analysis result, the degree of the heart rate of the user does not satisfy a preset heart rate level for the preset time period, for example, the change in the user's heart rate is too high, the server 140 may notify at least one of the user's terminal 120, the medical staffs terminal 130, the external server 150, and the guardian's terminal 160, which has generated a request, of the warning situation via a notification such as a pop-up window, a text message, an SNS message, or the like.

As shown in FIG. 19, when the level of the user's body temperature-specific heart rate does not satisfy a preset body temperature-specific heart rate level (S1920), the server 140 may further store the body temperature-specific heart rate analysis result for a predetermined time (S1930).

When the storage of the body temperature-specific heart rate analysis result is completed, the server 140 may further generate a request to transmit the body temperature-specific heart rate analysis result to at least one of the user's terminal 120, the medical staff's terminal 130, and the external server 150 (S1940). In addition, when the storage of the body temperature-specific heart rate analysis result is completed, the server 140 may generate a request to transmit the body temperature-specific heart rate analysis result to the terminal 160 of a guardian protecting the user's terminal 120.

When a command for transmitting the body temperature-specific heart rate analysis result is requested from at least one of the user's terminal 120, the medical staff s terminal 130, and the external server 150 (S1950), the server 140 may transmit the body temperature-specific heart rate analysis result to at least one of the user's terminal 120, the medical staffs terminal 130, and the external server 150, which has generated a request (S1960). In addition, when a command for transmitting the body temperature-specific heart rate analysis result is requested from the terminal 160 of the guardian, the server 140 may transmit the body temperature-specific heart rate analysis result to the terminal 160 of the guardian who has requested the analysis results.

Furthermore, when, as a result of the body temperature-specific heart rate analysis, the degree of the body temperature-specific heart rate of the user does not satisfy a preset body temperature-specific heart rate level for the preset time period, for example, a change in heart rate is too high according to the change in the user's body temperature, the server 140 may notify at least one of the user's terminal 120, the medical staffs terminal 130, the external server 150, and the guardian's terminal 160, which has generated a request, of the warning situation via a notification such as a pop-up window, a text message, an SNS message, or the like.

The server 140 may transmit breathing exercise analysis result, heart rate analysis result, and temperature-specific heart rate analysis result e, which are obtained by analyzing the breathing exercise state determination data OD2 output by an artificial intelligence model (AIM), the heart rate state determination data OD3 according to breathing exercise, and the body temperature-specific heart rate state determination data OD4 according to breathing exercise according to the input of breathing exercise data, heart rate data, and body temperature data, to at least one of the user's terminal 120, the medical staffs terminal 130, the external server 150, and the guardian's terminal 160, which had generated a request.

The server 140 may transmit an accurate breathing exercise analysis result for a breathing exercise status, an accurate heart rate analysis result, and an accurate body temperature-specific heart rate analysis result with respect to each user to at least one of the user's terminal 120, the medical staffs terminal 130, the external server 150, and the guardian's terminal 160, thus providing an accurate analysis service for the breathing exercise status for each user.

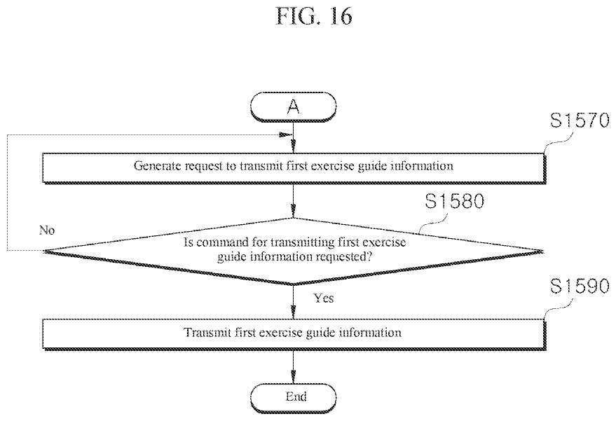

Referring to FIGS. 15 and 16, when a command for transmitting the breathing exercise analysis result is requested from at least one of the user's terminal 120, the medical staffs terminal 130, and the external server 150 (S1550), the server 140 may further generate a request to transmit first exercise guide information for strengthening the lungs of the user according to the breathing exercise analysis result to at least one of the user's terminal 120, the medical staffs terminal 130, and the external server 150, which has generated a request (S1570). In this case, when a command for transmitting the breathing exercise analysis result is requested from the guardian's terminal 160, the server 140 may further generate a request to transmit the first exercise guide information to the guardian's terminal 160.

When a command for transmitting the first exercise guide information is requested from at least one of the user's terminal 120, the medical staffs terminal 130, and the external server 150, which has generated a request (S1580), the server 140 may further transmit the guide information to at least one of the user's terminal 120, the medical staffs terminal 130, and the external server 150, which has requested the first exercise guide information (S1590). In this case, when the command for transmitting the first exercise guide information is requested from the terminal 160 of the guardian which has generated a request, the server 140 may further transmit the first exercise guide information to the terminal 160 of the guardian which has requested the first exercise guide information.

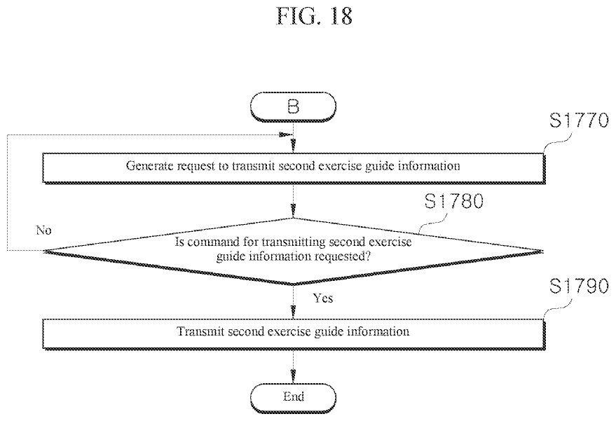

Referring to FIGS. 17 and 18, when a command for transmitting the heart rate analysis result is requested from at least one of the user's terminal 120, the medical staffs terminal 130, and the external server 150 (S1750), the server 140 may generate a request to transmit second exercise guide information for strengthening the heart of the user according to the heart rate analysis result to at least one of the user's terminal 120, the medical staffs terminal 130, and the external server 150, which has generated a request (S1770). In this case, when a command for transmitting the heart rate analysis result is requested from the guardian's terminal 160, the server 140 may further generate a request to transmit the second exercise guide information to the guardian's terminal 160.

When a command for transmitting the second exercise guide information is requested from at least one of the user's terminal 120, the medical staffs terminal 130, and the external server 150, which has generated a request (S1780), the server 140 may further transmit the second exercise guide information to at least one of the user's terminal 120, the medical staffs terminal 130, and the external server 150, which has requested the second exercise guide information (S1790). In this case, when the command for transmitting the second exercise guide information is requested from the terminal 160 of the guardian which has generated a request, the server 140 may further transmit the second exercise guide information to the terminal 160 of the guardian which has requested the second exercise guide information.

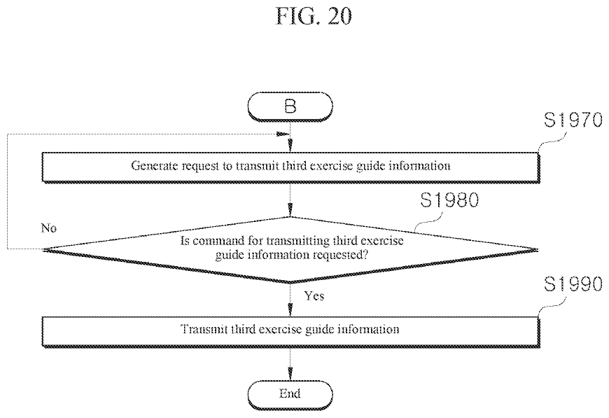

Referring to FIGS. 19 and 20, when a command for transmitting the body temperature-specific heart rate analysis result is requested from at least one of the user's terminal 120, the medical staffs terminal 130, and the external server 150 (S1950), the server 140 may generate a request to transmit third exercise guide information for strengthening the body temperature-specific heart state of the user according to the body temperature-specific heart rate analysis result to at least one of the user's terminal 120, the medical staffs terminal 130, and the external server 150, which has generated a request (S1970). In this case, when a command for transmitting the body temperature-specific heart rate analysis result is requested from the guardian's terminal 160, the server 140 may further generate a request to transmit the third exercise guide information to the guardian's terminal 160.

When a command for transmitting the third exercise guide information is requested from at least one of the user's terminal 120, the medical staffs terminal 130, and the external server 150, which has generated a request (S1980), the server 140 may further transmit the third exercise guide information to at least one of the user's terminal 120, the medical staffs terminal 130, and the external server 150, which has requested guide information (S1990). In this case, when the command for transmitting the third exercise guide information is requested from the terminal 160 of the guardian which has generated a request, the server 140 may further transmit the third exercise guide information to the terminal 160 of the guardian which has requested the third exercise guide information.

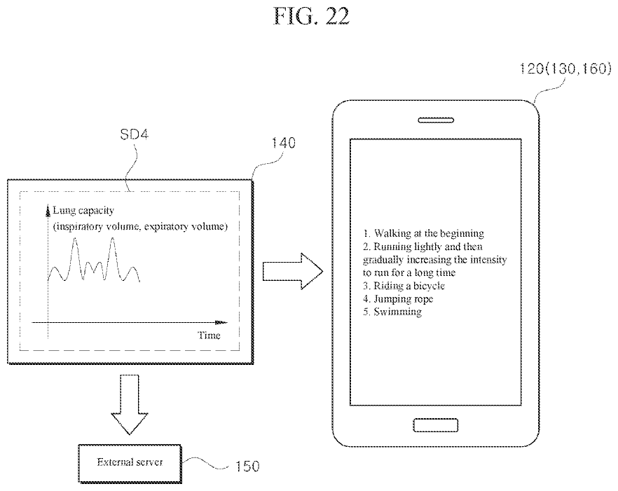

FIGS. 22 to 24 are diagrams illustrating, as an example, a process of transmitting exercise guide information according to the analysis result analyzed by the server of FIG. 1 to at least one of a user's terminal, a medical staffs terminal, an external server, and a guardian's terminal.

Referring to FIG. 22, the server 140 may transmit first exercise guide information for strengthening lungs of the user according to to breathing exercise analysis result information SD4 of the user to at least one of the user's terminal 120, the medical staff s terminal 130, the external server 150, and the terminal 160 of the guardian. In this case, the breathing exercise analysis result information SD4 may be values such as inspiratory volume, expiratory volume, lung capacity, and the like obtained by analyzing the user's breathing exercise process based on the magnetic sensor 111. In this case, the server 140 may determine that the user's inspiratory volume, expiratory volume, and lung capacity are not normal when they do not satisfy each preset reference value, and transmit the first exercise guide information for strengthening the lung condition corresponding thereto to at least one of the user's terminal 120, the medical staff's terminal 130, the external server 150, and the guardian's terminal 160. In this case, at least one of the user's terminal 120, the medical staffs terminal 130, and the guardian's terminal 160 may display the first exercise guide information on the screen. For example, at least one of the user's terminal 120, the medical staffs terminal 130, and the guardian's terminal 160 may display the first exercise guide information “1. Walking at the beginning, 2. Running lightly and then gradually increasing the intensity to run for a long time, 3. Riding a bicycle, 4. Jumping rope, 5. Swimming” on the screen. The external server 150 may receive and database at least one of the breathing exercise analysis result information SD4 and the first exercise guide information, and at least one of researchers and medical staffs may perform at least one of research and treatment on breathing by using at least one of the breathing exercise analysis result information SD4 and the first exercise guide information which has databased.

In addition, the server 140 may determine that the user's inspiratory volume, expiratory volume, and lung capacity are not normal when they do not satisfy each preset reference value, and transmit first examination guide information for suggesting lung examination and first food guide information for enhancing lung condition to at least one of the user's terminal 120, the medical staff's terminal 130, the external server 150, and the guardian's terminal 160.

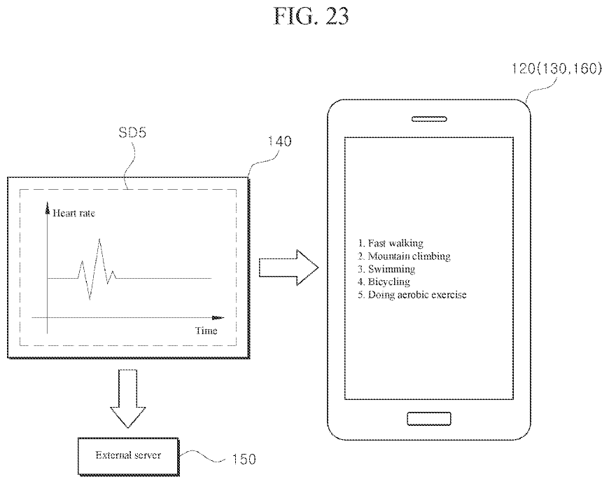

Referring to FIG. 23, the server 140 may transmit second exercise guide information for strengthening the heart of the user according to heart rate analysis result information SD5 of the user to at least one of the user's terminal 120 and the medical staff s terminal 130, the external server 150, and the terminal 160 of the guardian. In this case, the heart rate analysis result information SD5 may be a heart rate value obtained by analyzing the heart rate state of the user based on the heart rate sensor 112. In this case, when the user's heart rate value does not satisfy a preset reference value, the server 140 may determine that it is not normal, and transmit the second exercise guide information for strengthening the heart to at least one of the user's terminal 120, the medical staffs terminal 130, the external server 150, and the guardian's terminal 160. In this case, at least one of the user's terminal 120, the medical staffs terminal 130, and the guardian's terminal 160 may display the second exercise guide information on the screen. For example, at least one of the user's terminal 120, the medical staffs terminal 130, and the guardian's terminal 160 may display the second exercise guide information of “1. Fast walking, 2. Mountain climbing, 3. Swimming, 4. Bicycling, 5. doing aerobic exercise” on the screen. The external server 150 may receive and database at least one of the heart rate analysis result information SD5 and the second exercise guide information, and at least one of researchers and medical staffs may perform at least one of research and treatment on the heart rate by using at least one of the heart rate analysis result information SD5 and the second exercise guide information which has databased.

In addition, when the user's heart rate value does not satisfy a preset reference value, the server 140 may determine that it is not normal, and transmit at least one of second examination guide information for suggesting a heart examination and second food guide information for strengthening heart condition to at least one of the user's terminal 120, the medical staffs terminal 130, the external server 150, and the guardian's terminal 160.

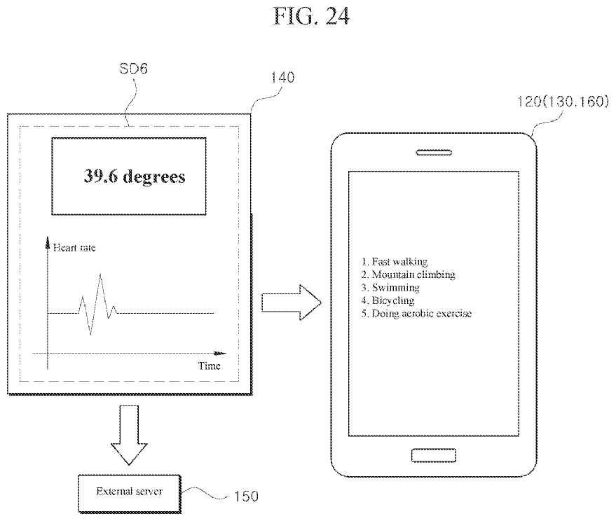

Referring to FIG. 24, the server 140 may transmit third exercise guide information for strengthening the heart condition of the user according to body temperature-specific heart rate analysis result information SD6 of the user to at least one of the user's terminal 120, the medical staffs terminal 130, the external server 150, and the guardian's terminal 160. In this case, the body temperature-specific heart rate analysis result information SD6 may be a heart rate value obtained by analyzing the user's heart rate state based on the heart rate sensor 112 and the user's body temperature value obtained by analyzing the body temperature state of the user based on the body temperature sensor 114. In this case, when the user's heart rate value for each body temperature does not satisfy a preset reference value, the server 140 may determine that it is not normal, and transmit the third exercise guide information for strengthening the heart condition to at least one of the user's terminal 120, the medical staffs terminal 130, the external server 150, and the guardian's terminal 160. The at least one of the user's terminal 120, the medical staffs terminal 130, and the guardian's terminal 160 may display the third exercise guide information on the screen. For example, at least one of the user's terminal 120, the medical staffs terminal 130, and the guardian's terminal 160 may display second exercise guide information “1. Brisk walking, 2. Mountain climbing, 3. Swimming, 4. Biking, 5. Doing aerobic exercise” on the screen. The external server 150 may receive and database at least one of the body temperature-specific heart rate analysis result information SD6 and the third exercise guide information, and at least one of researchers and medical staffs may perform at least one of research and treatment on the body temperature-specific heart rate by using at least one of the body temperature-specific heart rate analysis result information SD6 and the third exercise guide information which has databased.

Also, when the user's heart rate value for each body temperature does not satisfy a preset reference value, the server 140 may determine that it is not normal, and transmit third examination guidance information for suggesting a heart examination and third food guide information for strengthening a heart condition to at least one of the user's terminal 120, the medical staffs terminal 130, the external server 150, and the guardian's terminal 160.

As described above, the inventive concept may provide an analysis service for the sleep state of each user and a customized service for improving the sleep state of each user.

In addition, the inventive concept may monitor, store, and analyze a user's biological state in real time through various sensors of a patch-type sensor, and provide guide information for improving a sleep state.

In addition, the inventive concept may further include a vibrator that notifies when a period or position value of a signal obtained by various sensors of the patch-type sensor is out of a reference range. The vibrator may be included in the user's terminal, or may be included in at least one of a medical staffs terminal and a guardian's terminal.

In addition, the inventive concept may be configured as a mobile app and software to enable real-time monitoring of stored data and analysis results.

In addition, the inventive concept may manage a user's sleep and health by accurately measuring and recording the user's biological signals in real time through a patch-type subminiature sensor and analyzing the biological signals in various ways including AI.

In addition, the inventive concept may allow users and persons involved (medical staff, guardians, administrators of external servers) to monitor materials and analysis results stored in real time or after sleep through a network by installing a mobile app and SW on a mobile device including smart phone and a computer.

In addition, the inventive concept may improve results in various analyzes/evaluations using biological response information including the sleep analysis of a user.

In addition, the inventive concept may store data on biometric response information and later, improve results through management through analysis in various analyzes/evaluations using biometric response information including the sleep analysis of a user.

In addition, the inventive concept may provide an analysis service for breathing exercise for each user and a customized service for providing a guide for improving breathing exercise for each user.

In addition, the inventive concept may provide an analysis service of heart rate according to breathing exercise for each user, and may provide a customized service for providing a guide for improving heart rate for each user according to the breathing exercise.

In addition, the inventive concept may provide an analysis service of heart rate for each body temperature according to breathing exercise for each user, and may provide a customized service for providing a guide for improving a heart rate for each body temperature for each user according to the breathing exercise.

In addition, the inventive concept may improve the result by implementing various breathing exercise with game-type content and a display device.

In addition, the inventive concept may provide a monitoring and prediction system that stores data on the user's breathing exercise situation in real time and reflects the data on the next training through analysis.

In addition, the inventive concept may improve the radiation therapy effect of patients undergoing respiratory-gated radiation therapy. In addition, the inventive concept may reduce unnecessary radiation exposure to patients undergoing respiratory-gated radiation therapy. In addition, the inventive concept may provide a customized breathing exercise program during respiratory-gated radiation therapy.

In addition, the inventive concept may assist in increasing lung capacity and having regular breathing patterns in daily life by analyzing and implementing various training situation patterns through a virtual reality system.

In addition, the inventive concept may provide a breathing exercise system to prevent pneumonia that may occur as a postoperative sequelae. That is, the inventive concept provides a breathing exercise system for preventing pneumonia that may occur as a postoperative sequelae by expanding the lungs shrunk during surgery in a patient who has undergone general anesthesia surgery, as well as providing breathing exercise for respiratory-gated radiation therapy for lung cancer and liver cancer patients.

In addition, the inventive concept may implement breathing exercise through various video/audio devices such as mobile phones, tablets, computers, TVs, HMDs, and the like. In addition, the inventive concept may increase portability and convenience by allowing a virtual reality-based breathing exercise system to be cooperative with a mobile device such that a certain cycle of breathing is maintained. In addition, the inventive concept may provide an interface including game-type content capable of inducing motivation and setting various situations through a virtual reality-based breathing exercise system. In addition, the inventive concept may significantly increase the effects of various training through virtual reality.

At least one component may be added or deleted corresponding to the performance of the components shown in FIGS. 1 to 3, 6 to 14, and 21 to 24. In addition, it will be easily understood by those skilled in the art that the mutual positions of the components may be changed according to the performance or structure of the system.

Although it is described with reference to FIGS. 4, 5, 15, and 20 that a plurality of steps are sequentially performed, this is merely illustrative of the technical idea of the embodiment. Those of ordinary skill in the art to which this embodiment belongs may perform various modifications and variations such as changing the order described in FIGS. FIGS. 4, 5, 15, and 20 or performing one or more of the plurality of steps in parallel without departing from the essential features of the present embodiment, so that the inventive concept is not limited to chronological order in FIGS. 4, 5, 15, and 20.

Meanwhile, the disclosed embodiments may be implemented in the form of a recording medium storing instructions executable by a computer. The instructions may be stored in the form of program codes, and, when executed by a processor, perform operations of the disclosed embodiments by creating program modules. The recording medium may be implemented as a computer-readable recording medium.

The computer-readable recording medium includes all types of recording media capable of storing instructions decodable by a computer. For example, the computer-readable recording medium may include read only memory (ROM), random access memory (RAM), magnetic tape, magnetic disk, flash memory, optical data storage device, and the like.

As above, the disclosed embodiments have been described with reference to the accompanying drawings. Those skilled in the art to which the inventive concept belongs will understand that the inventive concept may be implemented in a form different from the disclosed embodiments without changing the technical spirit or essential features of the inventive concept. The disclosed embodiments are illustrative and should not be construed as limiting.

According to the above-mentioned embodiments of the inventive concept, it is possible to provide an analysis service for a sleep state for each user.

Furthermore, according to the above-mentioned embodiments of the inventive concept, it is possible to provide a customized service for improving a sleep state for each user.

Furthermore, according to the above-mentioned embodiments of the inventive concept, it is possible to improve the precision of sleep monitoring.

Furthermore, according to the above-mentioned embodiments of the inventive concept, it is possible to monitor breathing during sleep.

Furthermore, according to the above-mentioned embodiments of the inventive concept, it is possible to reduce accidents due to sleep apnea.

Furthermore, according to the above-mentioned embodiments of the inventive concept, it is possible to measure and record various bio-signals in real time.

Furthermore, according to the above-mentioned embodiments of the inventive concept, it is possible to improve the quality of sleep through real-time biosignal analysis.

Furthermore, according to the above-mentioned embodiments of the inventive concept, it is possible to improve the quality of sleep through biosignal analysis using AI.

Furthermore, according to the above-mentioned embodiments of the inventive concept, it is possible to provide an analysis service for breathing exercise for each user.

Furthermore, according to the above-mentioned embodiments of the inventive concept, it is possible to provide an analysis service of heart rate according to breathing exercise for each user.

Furthermore, according to the above-mentioned embodiments of the inventive concept, it is possible to provide an analysis service of heart rate for each body temperature according to breathing exercise for each user.

Furthermore, according to the above-mentioned embodiments of the inventive concept, it is possible to provide a customized service for providing a guide for improving breathing exercise for each user.

Furthermore, according to the above-mentioned embodiments of the inventive concept, it is possible to provide a customized service for providing a guide for improving a heart rate according to breathing exercise for each user.

In addition, according to the above-mentioned embodiments of the inventive concept, it is possible to provide a customized service for providing a guide for improving a heart rate for each body temperature according to breathing exercise for each user.

However, effects of the inventive concept may not be limited to the above-described effects. Although not described herein, other effects of the inventive concept can be clearly understood by those skilled in the art from the following description.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A server for providing health care services, comprising:
a communication unit configured to communicate with at least one of a user's terminal, a medical staffs terminal related to the user's terminal, an external server related to the user's terminal, and a patch-type sensor; and
a processor configured to control an operation related to provision of the health care services,
wherein the processor is configured to:
receive a biological signal of a sleeping user measured by the patch-type sensor;
determine whether a sleep state of the user satisfies a preset sleep state condition for a preset time period as an analysis result of analyzing the biological signal through an artificial intelligence model;
store the analysis result for the preset time period when the sleep state of the user does not satisfy the sleep state condition;
generate a request to transmit the analysis result to at least one of the user's terminal, the medical staffs terminal, and the external server when the storing of the analysis result is completed; and
transmit the analysis result to at least one of the user's terminal, the medical staffs terminal, and the external server which has generated the request when a command for transmitting the analysis result is requested from at least one of the user's terminal, the medical staffs terminal, and the external server.

2. The server of claim 1, wherein the processor is configured to:
further generate a request to transmit guide information for improving the sleep state of the user according to the analysis result to at least one of the user's terminal, the medical staffs terminal, and the external server which has generated a request when the command for transmitting the analysis result is requested; and
further transmit the guide information to at least one of the user's terminal, the medical staffs terminal, and the external server which has generated a request when a command for transmitting the guide information is requested from at least one of the user's terminal, the medical staffs terminal, and the external server which has generated a request.

3. The server of claim 2, wherein the processor is configured to determine whether a degree of breathing of the user satisfies a preset breathing level for the preset time period.

4. The server of claim 2, wherein the processor is configured to determine whether a degree of a heart rate of the user satisfies a preset heart rate level for the preset time period.

5. The server of claim 2, wherein the processor is configured to determine whether a degree of electrocardiogram of the user satisfies a preset electrocardiogram level for the preset time period.

6. The server of claim 2, wherein the processor is configured to determine whether a degree of a body temperature of the user satisfies a preset body temperature level for the preset time period.

7. The server of claim 2, wherein the processor is configured to determine whether a degree of a sleeping posture of the user satisfies a preset sleeping posture level for the preset time period.

8. The server of claim 2, wherein the communication unit further performs communication with a virtual reality device, and
wherein the processor is configured to:
receive breathing exercise information of the user who uses the virtual reality device measured by a magnetic sensor of the patch-type sensor;
determine whether a degree of breathing exercise of the user satisfies a preset breathing exercise condition for the preset time period as a result of analyzing the breathing exercise information through the artificial intelligence model;
store a breathing exercise analysis result for the preset time period when the degree of the breathing exercise of the user does not satisfy the breathing exercise condition;
generate a request to transmit the breathing exercise analysis result to at least one of the user's terminal, the medical staff's terminal related to the user's terminal, and the external server related to the user's terminal when storing of the breathing exercise analysis result is completed; and
transmit the breathing exercise analysis result to at least one of the user's terminal, the medical staffs terminal, and the external server which had generated the request when a command for transmitting the breathing exercise analysis result is requested from at least one of the user's terminal, the medical staffs terminal, and the external server.

9. The server of claim 8, wherein the processor is configured to:
further generate a request to transmit first exercise guide information for strengthening a lung condition of the user according to the breathing exercise analysis result to at least one of the user's terminal, the medical staffs terminal, and the external server which had generated the request when the command for transmitting the breathing exercise analysis result is requested; and
further transmit the first exercise guide information to at least one of the user's terminal, the medical staffs terminal, and the external server which had generated a request when a command for transmitting the first exercise guide information is requested from at least one of the user's terminal, the medical staffs terminal, and the external server which had generated the request.

10. The server of claim 9, wherein the processor is configured to:
further receive heart rate information of the user using the virtual reality device measured by a heart rate sensor of the patch-type sensor;
further determine whether a degree of heart rate of the user satisfies a preset heart rate level for the preset time period as a result of further analyzing the heart rate information through the artificial intelligence model;
further store a heart rate analysis result for the preset time period when the degree of heart rate of the user does not satisfy the heart rate level;
further generate a request to transmit the heart rate analysis result to at least one of the user's terminal, the medical staff's terminal related to the user's terminal, and the external server related to the user's terminal when storing of the heart rate analysis result is completed; and
transmit the heart rate analysis result to at least one of the user's terminal, the medical staffs terminal, and the external server which had generated the request when a command for transmitting the heart rate analysis result is requested from at least one of the user's terminal, the medical staffs terminal, and the external server.

11. The server of claim 10, wherein the processor is configured to:
further generate a request to transmit second exercise guide information for strengthening a heart condition of the user according to the heart rate analysis result to at least one of the user's terminal, the medical staffs terminal, and the external server which had generated the request when the command for transmitting the heart rate analysis result is requested; and
further transmit the second exercise guide information to at least one of the user's terminal, the medical staffs terminal, and the external server which had generated the request when a command for transmitting the second exercise guide information is requested from at least one of the user's terminal, the medical staffs terminal, and the external server which had generated the request.

12. The server of claim 11, wherein the processor is configured to:
further receive body temperature-specific heart rate information of the user who uses the virtual reality device measured by a body temperature sensor and the heart rate sensor of the patch-type sensor;
further determine whether a degree of body temperature-specific heart rate information of the user satisfies a preset body temperature-specific heart rate level for the preset time period as a result of further analyzing the body temperature-specific heart rate information through the artificial intelligence model;
further store a body temperature-specific heart rate analysis result for the preset time period when the degree of body temperature-specific heart rate of the user does not satisfy the body temperature-specific heart rate level;
further generate a request to transmit the body temperature-specific heart rate analysis result to at least one of the user's terminal, the medical staff's terminal related to the user's terminal, and the external server related to the user's terminal when storage of the body temperature-specific heart rate analysis result is completed; and
transmit the body temperature-specific heart rate analysis result to at least one of the user's terminal, the medical staffs terminal, and the external server which had generated the request when a command for transmitting the body temperature-specific heart rate analysis result is requested from at least one of the user's terminal, the medical staffs terminal, and the external server.

13. The server of claim 12, wherein the processor is configured to:
further generate a request to transmit third exercise guide information for strengthening a body temperature-specific heart state of the user according to the temperature-specific heart rate analysis result to at least one of the user's terminal, the medical staffs terminal, and the external server which had generated the request when the command for transmitting the temperature-specific heart rate analysis result is requested; and
further transmit the third exercise guide information to at least one of the user's terminal, the medical staffs terminal, and the external server which had generated the request when a command for transmitting the third exercise guide information is requested from at least one of the user's terminal, the medical staffs terminal, and the external server which had generated the request.

14. The server of claim 8, wherein the virtual reality device provides preset game-type content for each breathing exercise course to analyze the user's breathing exercise.

15. A server for providing health care services, comprising:
a user's terminal;
a medical staffs terminal related to the user's terminal;
an external server related to the user's terminal;
a patch-type sensor; and
a server configured to communicate with at least one of the the user's terminal, the medical staffs terminal, the external server, and the patch-type sensor,
wherein the server is configured to:
receive a biological signal of a sleeping user measured by the patch-type sensor;
determine whether a sleep state of the user satisfies a preset sleep state condition for a preset time period as an analysis result of analyzing the biological signal through an artificial intelligence model;
store the analysis result for the preset time period when the sleep state of the user does not satisfy the sleep state condition;
generate a request to transmit the analysis result to at least one of the user's terminal, the medical staffs terminal, and the external server when the storing of the analysis result is completed; and
transmit the analysis result to at least one of the user's terminal, the medical staffs terminal, and the external server which has generated a request when a command for transmitting the analysis result is requested from at least one of the user's terminal, the medical staffs terminal, and the external server.
